(19) United States
(12) Patent Application Publication
(10) Pub. No.: US 2009/0041824 A1
(43) Pub. Date: Feb. 12, 2009

(54) METHOD AND APPARATUS FOR COMPOSITE DRUG DELIVERY MEDICAL DEVICES

(75) Inventors: Greg Zugates, Cambridge, MA (US); David M. Dewitt, Allston, MA (US); Amar Kendale, Newton, MA (US); Irina Gitlin, Brookline, MA (US); Jeffrey Carbeck, Belmont, MA (US)

Correspondence Address: WOLF GREENFIELD & SACKS, P.C., 600 ATLANTIC AVENUE, BOSTON, MA, 02210-2206, US

(73) Assignee: Arsenal Medical, Inc., Watertown, MA (US)

(21) Appl. No.: 12/186,770

(22) Filed: Aug. 6, 2008

Related U.S. Application Data
(60) Provisional application No. 60/963,719, filed on Aug. 7, 2007.

(51) Int. Cl. A61F 2/00; A61K 47/30

(52) U.S. Cl. 424/423; 514/772.3

(57) ABSTRACT

A medical device and method for drug delivery employing a 3-dimensional pattern of a polymer support material (e.g., that is degradable after implant in a body), a drug associated with the polymer support material, and an adhesive that adheres the polymer support material and associated drug to body tissue. The adhesive may persist to maintain the device in a suitable position for a suitable time (e.g., until after the polymer support material begins to degrade to release the drug), and the drug may be arranged in discrete areas of the 3-dimensional pattern that are separated from each other. The pattern may be produced in whole or in part before deployment at a body site, or may be produced in whole or in part directly at a tissue surface.

This application claims the benefit of U.S. Provisional application 60/963,719, filed Aug. 7, 2007.

### BACKGROUND

1. Field of Invention

This invention relates to drug delivery devices for medical applications, e.g., devices that may be implanted in a human body to provide therapeutic effect, structural support and/or other functions.

2. Related Art

Implantable medical devices, such as stents, have been produced with incorporated drugs that are released over time to have a therapeutic or other desired effect, such as reducing thrombosis, promoting cell or other tissue growth, etc. For example, U.S. Pat. Nos. 7,070,590 and 5,490,962 disclose implantable drug delivery systems that contain a plurality of wells that each house an amount of drug that is released by degradation of the well structure.

### SUMMARY OF INVENTION

Aspects of the invention provide a medical device that is formed by the transfer, deposition or other printing of materials into a desired configuration for the purposes of providing (1) controlled drug delivery, (2) structural support to tissue, and/or (3) a barrier function to limit or prevent biological interactions with the tissue. As used herein, “printing” of a material includes any suitable technique or combination of techniques for providing one or more materials in desired locations, and may include roll coating, screen printing, drop printing (e.g., such as “ink jet” printing), transfer printing, UV or other light cured stereolithography, and others. A medical device may be formed in whole or in part before deployment at a tissue site (e.g., in a human patient), and/or may be formed in whole or in part at the tissue site.

In one illustrative embodiment, a medical device includes a 3-dimensional pattern of printed elements formed by a composite of a polymer support material, a drug, and an adhesive, e.g., carried on a surface of a backing material. The pattern of polymer/drug/adhesive may be deployed at a desired tissue site by placing the pattern of printed elements in contact with the tissue site, e.g., by pressing the backing material and associated pattern into contact with the tissue. In other embodiments, the pattern of printed elements may be formed directly onto a tissue site, eliminating any need for a backing material. The adhesive in the pattern may be sufficient to adhere the device, along with any backing material, to the tissue. The polymer may be associated with the drug, e.g., may be adjacent the drug, may encapsulate the drug by completely or partially surrounding a region of drug material, or may have the drug mixed in with the polymer, and may control release of the drug based on the degradation characteristics of the polymer. The drug release rate may be controlled, for example, based on the type of polymer material, the physical arrangement of the polymer (e.g., higher surface area features tend to degrade faster than lower surface area features), and/or the type or arrangement of adhesive in the pattern.

In one embodiment, a backing material that carries the 3-dimensional pattern may be made of a fast dissolving or otherwise degrading material so that after deployment of the device, the pattern of polymer/drug/adhesive is left alone at the tissue site. In an alternative embodiment, the backing material may be removed from the deployed pattern, e.g., by way of a release layer or other arrangement, leaving behind the pattern of polymer/drug/adhesive on the tissue surface. The pattern of printed elements and/or the backing material can serve as a means for altering the mechanical properties of the tissue, e.g., the pattern of polymer/drug/adhesive and/or the backing material may function as a stent, scaffold, prosthetic or other support, or provide selective barrier properties, e.g., to control material transport at the tissue interface.

In one aspect of the invention, the pattern of printed elements may include discrete areas of drug, e.g., wells or other relatively concentrated drug-carrying regions, that are separated from each other. The discrete areas may be formed in any suitable way, such as by encapsulating regions of drug in discrete, separated regions of polymer material. For example, the pattern of printed elements may include an array of multiple “islands” or discontinuous elements that each include polymer, drug and adhesive. For example, the drug may be mixed with the polymer and deposited in the desired pattern of discontinuous elements, with adhesive provided to the pattern thereafter. Alternately, the drug, polymer and adhesive may be mixed together, and then deposited to form the pattern. A solvent may be employed, e.g., to help maintain the drug, polymer and/or adhesive components in solution or otherwise provide the mixture with a suitable (e.g., relatively low) viscosity for printing. The pattern may be carried by a backing material and applied by a tissue site so that each of the elements in the array is individually adhered to the tissue site in a desired arrangement, e.g., corresponding to the arrangement of the pattern on the backing material. The discrete areas of drug may be formed in other ways, such as by providing individual, separated droplets or other areas of drug on a uniform sheet-like layer of polymer, providing isolated regions of drug between a pair of layers of polymer, and so on.

In one aspect of the invention, a drug delivery device is implantable in a body and includes a 3-dimensional pattern of a polymer support material (e.g., that is degradable after implant in a body), a drug associated with the polymer support material (e.g., such that portions of the drug are released as the polymer support material degrades), and an adhesive that adheres the polymer support material and associated drug to body tissue. The adhesive may persist for a suitable period, e.g., to allow tissue ingrowth and/or after the polymer support material degrades to release the drug, and the drug may be arranged in discrete areas of the 3-dimensional pattern that are separated from each other. For example, the discrete areas of drug may be separated from each other by gaps or voids in the polymer support material and/or the adhesive.

In one embodiment, the delivery device may include a backing material that carries the pattern of polymer support material, drug and adhesive and is arranged to support the polymer support material, drug and adhesive when the device is deployed at a body site. For example, the backing material may be a thin film that supports a continuous and/or discontinuous array of printed elements forming the 3-dimensional pattern. The backing material may be arranged to degrade faster than the polymer support material and/or adhesive. Thus, shortly after deployment of the device, only the 3-dimensional pattern of polymer, drug and adhesive may remain at the tissue site.

The device may take any suitable shape, before or after deployment, and may be adjusted in shape during deployment. For example, the device may have a sheet-like shape, and may be flexible so as to take on a tubular or other shape during deployment. In another embodiment, the device may have a more resilient or rigid structure so that the device may structurally support a tissue site at which the device is deployed.

In one embodiment, the 3-dimensional pattern may be formed by depositing polymer, drug, adhesive and/or solvent components in separate layers or other printed element arrangements. For example, the polymer support material may be formed in a first layer (e.g., on a backing material or onto a tissue site), the drug may be formed in a second layer on the first layer, and polymer support material may be formed in a third layer on the second layer. Adhesive may be formed in a fourth layer on the third layer, or directly onto the tissue site with the first layer formed on the adhesive layer. The printed elements that make up the 3-dimensional pattern are not limited to such a layered arrangement, and instead may be arranged in any suitable way. For example, the printed elements may have the same or different combination of polymer, drug, adhesive and/or solvent, and may have the same or different physical arrangement, and may have the same or different overall layout. As used herein, a “combination of A, B and/or C” is meant to refer to an arrangement having A only, B only, C only, A and B, A and C, B and C, or A, B and C. Also, a “physical arrangement” of a printed element refers to the physical size and shape of the printed element, not its location in the 3-dimensional pattern. A “layout” of printed elements refers to the location of printed elements in a pattern relative to other printed elements and the pattern as a whole.

In another embodiment, a solvent may be mixed with the polymer support material, drug and/or adhesive. The solvent may render the mixture a liquid or other printable form so that the 3-dimensional pattern can be more easily produced. After printing of the solvent/polymer/drug/adhesive mixture (e.g., to a backing material or tissue site), the solvent may be removed, e.g., by evaporation, absorption, diffusion, or other means.

In another aspect of the invention, a method for providing a delivery device includes providing a 3-dimensional pattern of polymer support material, drug and adhesive in an arrangement such that the drug is in association with the polymer support material, and the adhesive is arranged to adhere the pattern to body tissue. The drug may be arranged in discrete areas in the 3-dimensional pattern that are separated from each other. Also, the 3-dimensional pattern may be formed prior to deployment at a tissue site (in whole or in part), or may be formed directly onto the tissue site (in whole or in part). For example, the 3-dimensional pattern may be formed by printing polymer, drug and/or adhesive onto a backing material, and the 3-dimensional pattern of printed elements may be deployed at a tissue site thereafter. Alternately, polymer, drug and/or adhesive may be applied (e.g., in liquid form) to a tissue site in a desired arrangement so as to form the 3-dimensional pattern. In one embodiment, a portion of the 3-dimensional pattern may be formed directly onto the tissue site, and another portion of the 3-dimensional pattern may be pre-fabricated and deployed with the portion formed directly on the tissue site. For example, adhesive may be printed in a desired arrangement onto a tissue site, and thereafter an arrangement of polymer and drug may be deployed (e.g., with a backing material) onto the adhesive so that the adhesive adheres the polymer and drug arrangement to the tissue.

Methods for forming or otherwise providing a 3-dimensional pattern of polymer/drug/adhesive may be performed in accordance with various aspects of the invention described above. Thus, the 3-dimensional pattern may be formed by printing a plurality of printed elements that may have the same, or different, physical arrangement, and the same, or different, combination of polymer, drug and/or adhesive. For example, the 3-dimensional pattern may be formed by a plurality of printed elements arranged so that a plurality of voids separate discrete areas of drug. As one example, the 3-dimensional pattern may include a plurality of discontinuous regions of polymer support material and drug. Also, a solvent may be mixed with the polymer support material, drug and/or adhesive to form a mixture, and at least a portion of the 3-dimensional pattern may be formed by depositing the mixture.

These and other aspects of the invention will be apparent from the following description and claims.

### BRIEF DESCRIPTION OF THE DRAWINGS

Aspects of the invention are described below with reference to the following drawings in which like numerals reference like elements, and wherein:

FIG. 1 is a perspective view of an illustrative embodiment of a drug delivery device in accordance with aspects of the invention;

FIG. 2 is a perspective view of another illustrative embodiment of a drug delivery device in accordance with aspects of the invention;

FIG. 3 is a perspective view of drug delivery device deployed in a vascular application;

FIG. 4 is a perspective view of another illustrative embodiment of a drug delivery device arranged for deployment via catheter in accordance with aspects of the invention; and

FIG. 5 is a perspective view of the FIG. 4 embodiment with a deployment balloon in an inflated condition.

### DETAILED DESCRIPTION

It should be understood that aspects of the invention are described herein with reference to the figures, which show illustrative embodiments in accordance with aspects of the invention. The illustrative embodiments described herein are not necessarily intended to show all aspects of the invention, but rather are used to describe a few illustrative embodiments. Thus, aspects of the invention are not intended to be construed narrowly in view of the illustrative embodiments. In addition, it should be understood that aspects of the invention may be used alone or in any suitable combination with other aspects of the invention.

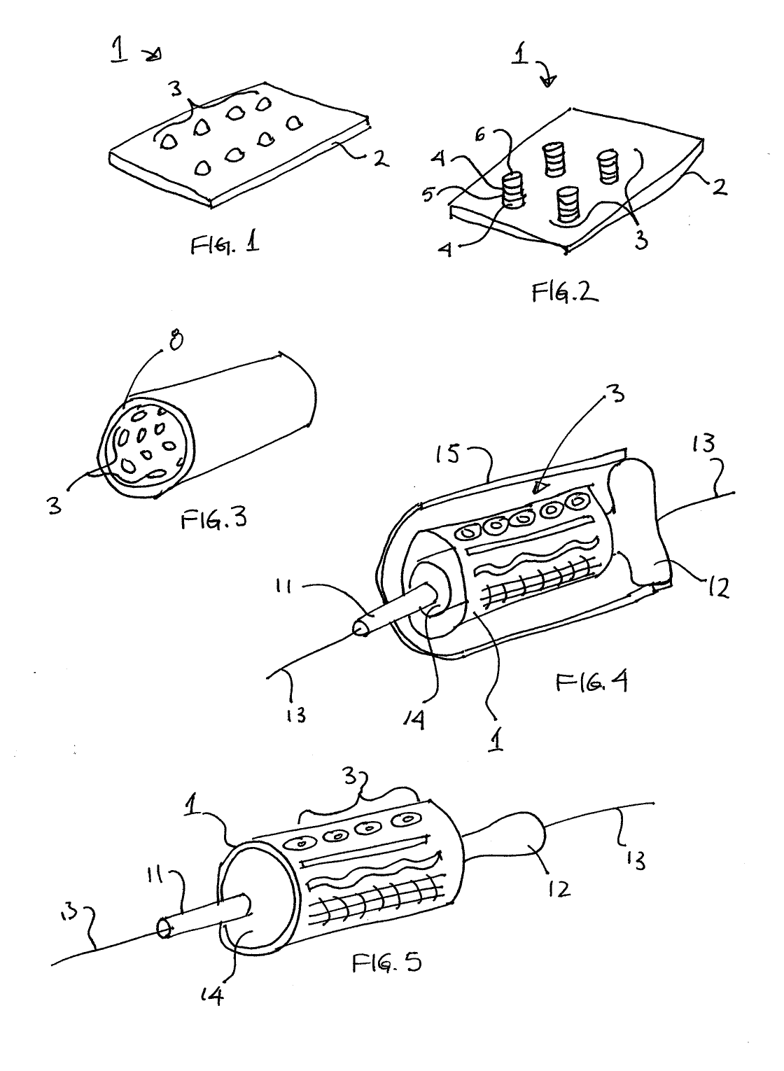

FIG. 1 shows an illustrative embodiment of a medical device in accordance with aspects of the invention. In this embodiment, the medical device 1 has a sheet-like shape that is implantable in an animal body (such as a human) and includes a backing material 2 in the form of a thin, flexible film, and an array or 3-dimensional pattern 3 of printed elements on a surface of the backing material 2. However, as will be understood from the description above and below, the medical device 1 is not limited to any particular shape, and may take any suitable configuration, such as a stent, graft, plug, patch, or other arrangement used with other types of implanted medical devices.

As will be discussed in more detail below, the pattern 3 may include components to provide a desired therapeutic or other effect, such as release of a drug, providing structural support to a tissue surface (which support may be temporary or permanent), providing a barrier function (e.g., to resist tissue ingrowth or cell migration, etc.), and/or others. The pattern 3 of printed elements may include up to four (or more) different components including a drug (i.e., any biologically active agent including one or more compounds), a polymer support material (i.e., one or more polymers, whether combined together or used separately, to support the drug and control its release rate), an adhesive (a material that adheres the device 1 to a tissue), and/or a solvent (a material that may hold the drug, polymer and/or adhesive in solution or provide a mixture with a viscosity suitable for printing to form the pattern).

In the example shown in FIG. 1, the pattern 3 of printed elements is in the form of a regular rectangular array of discontinuous elements (including an array of dots or circular regions), but any other suitable arrangement is possible, such as irregular arrays of discontinuous elements (randomly positioned dots or other regions), continuous patterns in which printed elements are connected to each other (such as grid or mesh patterns), combinations of continuous and discontinuous patterns (such as a grid pattern with dots of material located in spaces or voids in the grid), etc. For example, the pattern 3 may have the form of a bullseye (several concentric circles, ellipses, rectangles or other concentric shapes), a spiral, a series of parallel lines, a regular lined grid (like the printed grid on standard graph paper), multiple connected or unconnected shapes (such as a repeated pattern of circles, rings, diamonds, stars, blobs, etc.) and others. Thus, the pattern 3 shown in FIG. 1 is only one illustrative example of the different types of patterns 3 that may be employed with aspects of the invention. Also, each element or region in the pattern 3 need not include all components included in the pattern, e.g., each element need not necessarily include polymer, drug and adhesive. Instead, the pattern 3 may be arranged so that at least some areas of the pattern do not include one or more components. For example, the pattern 3 may have a regular lined grid form with polymer and adhesive located in the lines of the grid, yet the drug areas may be located only at the intersection points of the grid lines. In this way, discrete areas of drug may be located at the grid line intersections, and the discrete areas of drug may be separated by voids in the pattern 3.

Thus, the pattern 3 may be formed by a plurality of printed elements, which may each include polymer support material, drug, adhesive and/or solvent. Also, printed elements may have the same, or different, combination of polymer support material, drug, adhesive and/or solvent, and may have the same, or different, physical arrangement. For example, the pattern in FIG. 1 may be formed by mixing polymer, drug, adhesive and/or solvent together, and then printing the mixture to form printed elements in the arrangement shown in FIG. 1. In the pattern 3 of FIG. 1, the printed elements have the same combination of polymer support material, drug, adhesive and/or solvent, and have the same physical arrangement. Alternately, a pattern 3 may be formed by printing a plurality of separate printed elements that together form the pattern. For example, sequential printing steps can be performed to build a layered structure for the pattern 3, e.g., as shown in FIG. 2, where it is desirable to sequentially and separately print the polymer support material, drug, and/or adhesive. In the embodiment of FIG. 2, printed elements may be arranged in stacks with a first set of printed elements of polymer support material 4 initially deposited on the backing material 2, followed by a second set of printed elements of drug 5, then a third set of printed elements of polymer support material 4 is formed over the drug, and a fourth set of printed elements of adhesive 6 is formed on the upper layer of polymer 4. Such a technique may be particularly useful to controllably release hydrophilic drugs (e.g., proteins, antibodies, growth factors, peptides, nucleic acids) that will not otherwise be easily mixed and distributed in a hydrophobic polymer. Thus, in the FIG. 2 embodiment, the printed elements have different combinations of polymer support material, drug, adhesive and/or solvent (each printed element includes only polymer, only drug, or only adhesive), yet have the same physical arrangement and are formed using the same layout. Furthermore, such an arrangement can be useful for patterning two separate polymers with different physicochemical properties so that the drug is released preferentially in a certain direction (toward the tissue or away from it). For example, the first lower layer of polymer 4 nearest the backing material 2 in FIG. 2 may be different from the second upper layer of polymer 4 above the drug 5, and thus have different properties, such as permeability, resorption rate, hydrophobicity, etc. Thus, the drug 5 may be released in a desired direction, whether away from the tissue site or towards the tissue.

Although deposition of the printed elements may be made in the same, registered layout (e.g., one layer on top of a prior layer as shown in FIG. 2), printed elements may be formed using the same overall layout, but in an unregistered way (e.g., one layer or set of printed elements partially overlapping and/or next to another prior layer). Alternately, printed elements may be formed using different layouts. Thus, the printed elements are not limited to having any particular shape, size, location or other arrangement in relation to other printed elements in a pattern. For example, the pattern 3 in FIG. 2 may be modified so that the first printed element (a layer of polymer 4) is formed as a single continuous film, on which discrete printed elements of drug 5, polymer 4 and adhesive 6 are deposited like that shown in FIG. 2. Of course, those of skill in the art will appreciate that a variety of other alternatives are possible, such as forming both the polymer 4 layers as a single continuous film, yet forming the drug 5 and adhesive 6 in discrete areas, and so on. In another illustrative embodiment in which the pattern 3 has a regular lined grid arrangement, a first set of printed elements forming vertical lines of the grid may include polymer only, a second set of printed elements forming horizontal lines of the grid may include adhesive only, and a third set of printed elements in the form of dots may include only drug and be printed at intersections between the vertical and horizontal lines of the grid. Thus, a pattern 3 may be formed by a group of printed elements that are formed in separate printing operations, and the printed elements may have different sizes, shapes and/or positions in the pattern. Of course, the pattern 3 may include printed elements having the same size, shape and/or location, the printed elements may include one or more components (polymer, drug, adhesive and/or solvent), or any other suitable arrangement.

In other embodiments, the pattern 3 may be formed by a combination of printing mixed compounds (e.g., printing a mixture of polymer, drug, adhesive and/or solvent) and individual compounds (e.g., printing polymer, drug, adhesive and/or solvent alone). For example, a drug/polymer mixture may be printed in a desired arrangement on a backing material 2 (e.g., in an arrangement like that in FIG. 1), followed by printing of an adhesive on/near the printed drug/polymer in the same or a different arrangement. In this example, a solvent may be used with the drug/polymer mixture and/or with the adhesive. In some embodiments, such as those including patterns 3 of discontinuous elements, adhesive should be associated with each element to adhere the element to the tissue surface. Also, the adhesive may be used to adhere the printed elements to the backing material 2, e.g., in the arrangements of FIGS. 1 and 2, adhesive may be provided to adhere the polymer/drug to the backing material 2. In addition, the adhesive may be used to control the drug release rate, e.g., by controlling the degradation rate of the polymer 4. The adhesive may control the degradation rate of the polymer 4 by reducing the surface area of the polymer that is exposed to body fluids and/or by controlling a diffusion rate through the adhesive. The adhesive may be a spontaneous adhesive (e.g., a cyanoacrylate, or polyisocyanate) or an activatible adhesive (e.g., acrylate-, methacrylate-, acrylamide-based chemicals). Also, the adhesive may be absorbable or otherwise degradable in a body, e.g., by providing suitable additives to the adhesive.

Any suitable printing techniques can be used to deposit polymer-drug-adhesive-and/or-solvent materials to form a desired pattern 3. Some suitable techniques may include micro-contact, pad, offset lithography, flexography, rotogravure, transfer printing, dip-pen lithography and various forms of inkjet printing (e.g., thermal, piezoelectric, continuous). Using these techniques, the shape and design of the printed elements can be varied and optimized for each application, e.g., the size, pitch, and/or density of printed elements can be varied and optimized for a given application. The solvent may be used to aid in the printing process, and may be removed after formation of the pattern 3, e.g., by evaporation or other techniques. Alternately, the solvent may be present at deployment of the device 1 at a tissue site, e.g., where the solvent is intended to have a therapeutic effect.

The polymer support material can be made of any suitable material, such as natural or synthetic polymers, or various combinations, such as glycosainioglycans (e.g., hyaluronic acid, heparin, chondroitin sulfate), proteins (e.g., collagen, gelatin, elastin, albumin), sugars (e.g., dextran, agarose, chitosan, carboxymethyl cellulose), lipids, polyesters (PGA, PLA, PLGA, PCL, PHB), polyanhydrides, poly(ortho esters), polycarbonates, polyvinyl alcohol, polyvinyl acetate, polyvinyl pyrrolidone, polyethyleneglycol, polypropylene glycol, and copolymers of any these materials.

Likewise, the backing material 2 may take any suitable form, and include any suitable material or combination of materials, depending on the function intended to be performed. For example, the backing material 2 can be made of natural or synthetic polymers, or various combinations, such as glycosaminoglycans (e.g., hyaluronic acid, heparin, chondroitin sulfate), proteins (e.g., collagen, gelatin, elastin, albumin), sugars (e.g., dextran, agarose, chitosan, carboxymethyl cellulose), lipids, polyesters (PGA, PLA, PLGA, PCL, PHB), polyanhydrides, poly(ortho esters), polycarbonates, polyvinyl alcohol, polyvinyl acetate, polyvinyl pyrrolidone, polyethyleneglycol, polypropylene glycol, and copolymers of any these materials. The backing material 2 can be fabricated by a number of methods, e.g., those that are typically used to make thin polymer films, such a solvent casting (with or without a porogen to introduce and control porosity of the backing material 2) or melt pressing/extrusion. In one illustrative embodiment, the backing material 2 is made of flexible material(s) that allow it to conform to a tissue surface upon application of the medical device to a tissue site. The pattern 3 associated with the backing material 2 may also be arranged to provide a desired flexibility, allowing the device 1 to closely conform even with uneven or convoluted surfaces.

In one aspect of the invention, the backing material 2 may dissolve/degrade quickly (e.g., on a time scale of seconds to hours once the device 1 is deployed at a tissue site) to leave a pattern 3 of printed elements on the tissue surface. Thus, the backing material 2 may be used in this embodiment as only a carrier to deploy the pattern 3 at the tissue site, persisting long enough to allow transport of the pattern 3 to a tissue site and positioning onto the tissue surface.

In another embodiment, the backing material 2 may degrade slowly, e.g., acting as a barrier. This barrier function could be useful in a number of instances such as colon cancer (to prevent tumor ingrowth), arterial disease (to prevent stenosis and provide support to the vessel wall), GI ulcers (to protect ulcers from rupture), and to control upper GI bleeding (causing hemostasis and protecting the lesion to limit recurrent bleeding).

In another aspect of the invention, the backing material 2 may be removed after deployment of the pattern 3, leaving behind only the pattern 3 of printed elements at the tissue site. For example, the adhesive 6 used to adhere the pattern 3 to tissue may have a higher bonding strength to the tissue than the mechanism by which the pattern 3 is adhered to the backing material 2. This may allow the backing material 2 to be pulled from the pattern 3 after the pattern 3 is adhered to the tissue. In another embodiment, the backing material 2 may include a release layer, e.g., a thin, readily absorbable sheet material, that allows a thicker sheet portion of the backing material 2 to be removed after deployment of the pattern 3.

The backing material 2 can be made in any suitable shape, whether in sheet or film form, or other three dimensional shapes. For film arrangements, the length and width of the film can be tailored to cover surfaces 1 mm² and larger. The thickness of the film may be in the size range of 10 nm-1 cm, with an optimal thickness dependent on the intended anatomical placement and device functions. For a tubular structure, the backing material 2 can be fabricated to sub-millimeter diameters and higher. Also, the sheet, tube or other shape could be slotted, spiral cut or otherwise configured to facilitate its packaging and/or deployment.

The backing material 2 material can be crosslinked to slow down, or control, its dissolution rate, particularly for hydrophilic materials. In these cases, a total dissolution or degradation time may be between a few seconds to several weeks, but may be more. For degradable hydrophobic materials, the appropriate time range for degradation may range from a day to a year, although other arrangements are possible. In addition to spontaneous degradation, the backing material 2 can be designed from materials that will degrade or dissolve in response to external, controlled stimuli or internal, biological stimuli, such as pH change, light, heat, ionic strength, mechanical effect (e.g., abrasion), enzymes, chemicals, or consumption of an activating agent (e.g., by drinking, inhaling, or topical administration).

Although the pattern 3 of printed elements may be designed to act as a reservoir for drug loading and controlled release, the backing material 2 may also contain drugs. The drug(s) may be blended homogeneously throughout the film and released as the materials degrades and/or by Fickian diffusion through the backing material 2, for example. In another embodiment, the backing material 2 can be fabricated in layers, with each layer composed of different materials and containing/releasing drug(s). In this embodiment, some of the backing material 2 layers can be removed, leaving behind other layers along with the pattern 3 of printed elements, as in the release layer embodiment described above.

Devices in accordance with aspects of the invention may be deployed or formed at any suitable tissue site. FIG. 3 shows an illustrative embodiment in which a pattern 3 of printed elements are deployed on the interior surface of a vessel wall 8. In this illustrative embodiment, the device 1 includes a fast-dissolving backing material 2 and a pattern 3 of printed elements in the form of a matrix of dots that are deployed on the endoluminal surface of a blood vessel. In the view shown in FIG. 3, the backing material 2 has dissolved, and the vessel endothelial surface has received the pattern 3 of printed elements. In another embodiment including a slow dissolving/degrading backing material 2, the backing material 2 may provide mechanical support to the vessel until the backing material 2 itself loses mechanical integrity due to the dissolution/degradation. Although the embodiments above are shown with the pattern 3 and backing material 2 in a flat sheet-like form, the pattern 3 and backing material 2 may be arranged in a tubular shape, e.g., for deployment in an application like that in FIG. 3, a partial spherical shell shape, a plug arrangement (like that used for hernia repair), or any other suitable arrangement. A “sheet” may be flat, take a cylindrical form, frustoconical form, a partial spherical form or any other shape.

Although many of the embodiments described above are formed by printing a pattern 3 onto a backing material 2, printing may be used to create a patterns of printed elements directly on a tissue surface or otherwise at a tissue site. (As used herein, a tissue site may include any suitable tissue or combination of tissues, including muscle, bone, cartilage, tendon, skin, and/or any other material found in an animal body.) For example, in the arrangement shown in FIG. 3, the pattern 3 of printed elements, including polymer/drug/adhesive, may be formed directly onto the endoluminal surface of the blood vessel. As with the embodiments described above, the pattern 3 may be formed by applying a mixture of polymer, drug, adhesive and/or solvent, and/or by applying individual components to form the pattern 3. As with all aspects of the invention, aspects may be used alone and/or in any suitable combination with other aspects of the invention. Thus, in one embodiment, a pattern 3 of printed elements that is formed on a backing material 2, and then deployed at a tissue site may be used in combination with printed elements that are formed directly on/at the tissue site. For example, an adhesive may be applied to the vessel wall alone in a desired arrangement, and thereafter an arrangement of polymer/drug carried on a backing material 2 may be applied to the adhesive. Printing of pattern 3 components onto a tissue site may be performed, for example, by a transfer stamp process in which a stamp having raised surfaces in a desired arrangement and carrying the component (e.g., polymer, drug, adhesive and/or mixtures of any two components with or without a solvent) transfers the component(s) to the tissue site by contact transfer. Other printing techniques are possible, including those mentioned above.

Some of the tissue surfaces that are of interest to deploy a medical device in accordance with aspects of the invention include the vasculature (arteries, veins, capillaries), GI tract (esophagus, stomach, intestines), pulmonary system (lungs, bronchioles, trachea, nasal passages, sinuses, mouth), urological system (bladder, urethra, tubules), lymphatic system (lymph ducts, vessels, capillaries, nodes), skin (i.e., topical), eye, surgically incised tissue surfaces and on surfaces for which post-surgical adhesions are to be prevented.

Deploying a medical device in accordance with aspects of the invention may be challenging in some applications, particularly if the pattern of printed elements are to be shielded from other biological surfaces or fluids while in transit to the target site. For example, to deploy a device in the coronary artery, it may be desirable to insert the device in the femoral artery and advance it through the vasculature to the target tissue site. While in transit, it may be necessary to protect the device from contact with the blood and other vascular surfaces prior to reaching the tissue site, especially if an adhesive included in the pattern 3 sets on contact with blood or tissue (e.g., cyanoacrylate). In this instance and for deploying the device on the surface of a vessel lumen (e.g., vasculature, GI tract, urological tracts), a combination balloon catheter can be used as shown in FIGS. 4 and 5.

FIG. 4 shows a device 1 having a backing material 2 and pattern 3 formed on an exterior surface of the backing material 2. (The device 1 in this embodiment shows several different pattern 3 arrangements, including concentric circles, straight and wavy lines, and a grid form.) The device 1 may be arranged in accordance with any of the suitable arrangements described above. The device 1 is mounted on a catheter 11, which in this embodiment includes a sealing balloon 12, a guidewire 13, a deployment balloon 14 and a sheath 15. The device 1 may be covered in whole or in part by the sheath 15, e.g., to help prevent contact between an adhesive in the pattern 3 and tissue surfaces before deployment. With the device 1 in place over the deployment balloon 14 and the sheath 15 covering the device 1, the sealing balloon 12 may be inflated so that the balloon 12 engages the interior surface of the sheath 15, preventing the passage of fluid or other materials through the distal opening of the sheath 15 and maintaining the sheath 15 in place over the device 1.

Once the device 1 mounted on the catheter 11 is positioned at a desired tissue site, the sheath 15 (if present) may be withdrawn (e.g., by deflating the sealing balloon 12 and pulling the sheath 15 proximally relative to the device 1). The deployment balloon 14 may then be expanded, pressing the pattern 3 into contact with the interior surface of the vessel wall, as shown in FIG. 5. Contact of the pattern 3 with the vessel tissue may cause adhesive in the pattern 3 to adhere to the vessel, thereby adhering the device 1 to the vessel wall. With the device 1 deployed at the tissue site, the deployment balloon 14 may be deflated, and the catheter 11 withdrawn.

Other methods to protect the pattern 3 of printed elements prior to deployment may be used, including the presence of a fast-degrading conformal sacrificial covering layer over the device 1; a sealing balloon that fully or partially encapsulates the pattern 3 of printed elements; two or more balloons located at one or both sides of the device 1 to provide redundant sealing protection and/or device centering; and an actuatable sacrificial covering layer (e.g., a thermally, optically, or electromagnetically activated material that is selectively degraded or otherwise exposes the pattern 3 to the tissue site upon activation).

Some advantages that may be provided by some aspects of the invention include:
Allows for the application of a pattern of polymer and drug on a tissue surface. This allows for the pattern of drug and polymer to be tailored to the desired treatment, since different tissue sites and/or conditions may warrant different patterns for drug release.
Drug release can be controlled by choice of polymer, adhesive and method of fabrication. Since drug release can be modified based on several different factors, the drug release can be relatively easily modified to suit a particular treatment. For example, a particular drug may be effectively encapsulated only by a particular polymer or class of polymers. However, the particular polymer(s) may have a degradation rate that is faster than desired. Aspects of the invention allow for modification in the arrangement of the polymer (e.g., thickness, pattern arrangement, etc.) as well as the arrangement or type of adhesive to help control drug release. For example, the drug may be encapsulated in a way like that shown in FIG. 2 with the suitable polymer above and below the drug, or like that in FIG. 1 in which the drug is dissolved in or otherwise mixed with the polymer. However, the arrangement may be modified, e.g., by providing a film of adhesive or another relatively slow degrading polymer over the polymer/drug regions, and by arranging the backing material 2 to degrade at a desired rate.
Directional drug release may be accomplished by layering with different polymers; drugs can be delivered locally to the tissue and/or released from the tissue surface for systemic treatment. For example, a particular drug may be more effective when released toward the tissue surface to which the pattern 3 is adhered. In this case, an arrangement like that in FIG. 2 may be used in which a slow degrading and/or low permeability polymer (with the permeability being determined based on a rate at which the drug may diffuse through the polymer to be released) may be used as a lower layer nearest the backing material 2, and a faster degrading and/or higher permeability polymer may be used as an upper layer nearest the tissue surface so that drug is preferentially released toward the tissue surface. In another application, it may be preferable to release drug in a direction away from the tissue surface. In this case, the polymer layers may be reversed in relation to that described above, with the faster degrading and/or higher permeability polymer positioned away from the tissue surface.
Layering polymer and drug may be an effective means of encapsulating and obtaining controlled release of hydrophilic drugs (e.g., peptides, antibodies, growth factors, nucleic acids), e.g., using an approach like that in FIG. 2.
Each printed element in a pattern can be made with different drugs, polymers, and/or adhesives so that multiple drugs can be released at different rates; the device 1 can effectively act as a “pharmacy” for treating one or multiple diseases. Since the pattern 3 of polymer/drug/adhesive can be made using printing techniques, the pattern 3 can include any suitable number or combination of components in any physical arrangement, such as two or more drugs, polymers and/or adhesives deposited in different pattern arrangements. Thus, if a pattern 3 is to include two different drugs, and each drug is better encapsulated by a different polymer, the pattern 3 can be made using the different polymers, each associated with a respective drug and physically arranged to best release the drug.
The device 1 may be arranged to include little material overall after deployment, which minimizes the surface area of device 1 that contacts the tissue and other bodily fluids. This may be important for patterns 3 that are printed on a blood vessel and contact the blood (less material is better for blood compatibility, maintaining vessel wall permeability, and preventing side branch occlusion).
The ability to deliver small, discrete printed elements may be important in the event that a printed element in a pattern dislodges from the tissue. For example, in vascular applications, printed elements in a pattern 3 (e.g., like that in FIG. 1 or 2) can be made small enough so that they will not occlude a blood vessel if they are dislodged and travel in the blood stream.
The pattern of printed elements may be multi-functional providing both mechanical and barrier properties, as well as drug delivery capabilities. For example, the polymer, adhesive and/or the backing material may be arranged to provide physical support to the tissue site to which the device 1 is adhered. In one embodiment, the device may function as a stent, helping to prop open a vessel. In other arrangements, the polymer, adhesive and/or backing material may provide a barrier function, helping to resist transfer of cells, drugs, nutrients and/or other materials.

Potential applications for embodiments in accordance with aspects of the invention include:

Cardiovascular
Atherosclerosis, vulnerable plaque

Gastrointestinal
Upper GI bleeding
Ulcers
Acid-reflux disease
Cancer (colorectal, stomach, esophagus)

Urological

Respiratory
Asthma
Sinus infection
Oral hygiene

Neurological
Pain control

Skin/Topical
Nicotine addition
Birth Control
Pain control
Anti-fungal

Ocular
Macular degeneration
Glaucoma
Infection (e.g., conjunctivitis)

### Aspects of the Invention as Specifically Applied to the Gastrointestinal (GI) System

Diseases and conditions of the gastrointestinal tract pose challenges that are substantially different from those encountered in other systems. Specifically, there are characteristics of gastrointestinal organs, tissues, and cell that are wholly unique to this system. These unique characteristics result in a drastically different chemical and biological environment. Aspects of the invention may require careful design to ensure biological compatibility.

Both non-structural and structural needs exist in the GI system. Furthermore, the specific nature of disease presentation requires appropriate selection of therapeutic agents (drugs, biologics, peptides, etc.). A series of representative applications and corresponding device embodiments is described below.

Non-Structural Applications

Upper GI bleeding: may be caused by ulcers (peptic, gastric, duodenal, esophageal, etc.), varices (esophageal, gastric, etc.), gastroduodenal erosions, esophagitis, etc. A primary function of a medical device in accordance with aspects of the invention for this application may be to locally deliver therapeutic agents that facilitate healing of the mucous membrane via release of pro-coagulation agents, vaso-active agents, sclerosing agents, antibacterial drugs and/or via local control of acid concentration using antacids, PPI or H2 antagonists. In many cases, the backing material 2 for these applications may be fast-degrading. The backing material 2 and pattern 3 may be deployed by catheter (e.g., as described in connection with FIG. 3 above) and the device 1 may be expandable to accommodate an esophageal diameter range of 8 to 65 mm (preferably 35-50 mm), a stomach spherical diameter range of 50 to 200 mm, a small intestinal diameter range of 8 to 100 mm (preferably 25-30 mm), and/or a large intestinal diameter range of 10 to 100 mm, preferable 50 to 100 mm. The backing material 2 may have a thickness ranging from 10 nm to 20 mm (preferably 25 um to 2.5 mm). The pattern 3 may be arranged to have an openness (i.e., a ratio of voids containing no polymer, drug or adhesive, to the total surface area of the device) of from 95% to 0% (5% to 100% coverage) of the target lumen. Some possible geometries for the device 1 include spiral cut tubes, slotted tubes, flat sheets, radial bellows, and longitudinal bellows.

Drugs used with the device may include one or more of:

Pro-coagulation agents such as:
fibrinogen, thrombin, or both (i.e. fibrin glue)

Vaso-active agents such as:
epinephrine

Sclerosing agents such as:
ethanol, polidocanal, sodium tetradecyl sulfate

Proton-pump inhibitors such as:
Omeprazole, Lansoprazole, Esomeprazole, Pantoprazole, Rabeprazole

Antibiotics such as:
Erythromycin, Ampicillin, Amoxicillin, Tetracycline, Metronidazole

Antacids such as:
H-2 receptor antagonists, Cimetidine, Ranitidine, Famotidine

Ulcers (Non-Bleeding)

A primary function of a medical device in treating non-bleeding ulcers may be to cover the site, prevent irritation by acid, and/or promote healing of the mucous membrane. Arrangements similar to those described above may be used for treating non-bleeding ulcers.

Varices (Non-Bleeding)

A medical device in accordance with aspects of the invention may provide controlled local release of the pharmaceutical agents that are commonly used in treatment of varices. Arrangements similar to those described above may be used for treating non-bleeding varices. In addition non-selective beta blockers, such as propranolol, timolol, and nadolol, may be used.

Acid-Reflux Disease

A medical device in accordance with aspects of the invention may provide controlled local release of the pharmaceutical agents that are commonly used in reducing the acid secretion in the GI tract. Arrangements similar to those described above may be used for treating acid reflux. Also, alginic acid, prokinetics, Cisapride, or Sucralfate may be included with the device.

A device may also be used in a protective application, e.g., for GI bleeding and acid-reflux disease the device may serve to deliver therapeutic agents and seal the site of the bleeding and serve as a barrier between the site and stomach acids. For non-bleeding ulcers and varices, the device could deliver therapeutic agents and protect the site from erosion and abrasion.

Structural Applications (Load-Bearing):

Cancer (Colorectal, Stomach, Esophagus)

A medical device in accordance with aspects of the invention may provide patency and support to the lumens of the GI tract and provide local drug delivery of the anti-cancer agents. Adhesive on the medical device may secure the device in place and prevent its migration in the lumen. In addition to the load-bearing structural function, the medical device may also provide a barrier function. A rigid or semi-rigid mechanical structure may be provided by one or more layers of the backing material 2, adhesive and/or polymer included in the device. A conformal external (abluminal) layer, e.g., made of a hydrogel, may be used to distribute stress and protect the lining of the tract. The conformal material may be arranged in such a way to fill the unpatterned space on the abluminal surface, thereby fully enabling adhesion of the pattern 3 of printed elements to the lumen wall. Rigidness of the device 1 may be contoured for the specific application (e.g., the device may be more compliant at the ends of the device relative to a center section). In addition to the use of adhesive, other retention features (such as hooks or sutures) may be included to prevent migration of the device. The device 1 may also include anti-proliferative agents, or anti-migratory agents. Also, the device may provide patency to the esophagus as well as barrier protection of varices, if present.

As used herein, “and/or” is meant to reflect the use of each individual element alone and any combination of elements. For example, a feature that includes “A, B and/or C” may include A only, B only, C only, A and B, A and C, B and C, or A, B and C. Also, a “combination of A, B and/or C” is meant to refer to an arrangement having A only, B only, C only, A and B, A and C, B and C, or A, B and C.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A drug delivery device that is implantable in a body, comprising:
a 3-dimensional pattern of:
a polymer support material;
a drug associated with the polymer support material, the drug being arranged in discrete areas of the 3-dimensional pattern that are separated from each other; and
an adhesive to adhere the polymer support material and associated drug to body tissue.

2. The device of claim 1, wherein the 3-dimensional pattern is formed by a plurality of printed elements each comprising polymer support material, drug and/or adhesive.

3. The device of claim 2, wherein the 3-dimensional pattern is formed by a plurality of printed elements each comprising polymer support material, drug and adhesive.

4. The device of claim 2, wherein the 3-dimensional pattern includes a plurality of printed elements that have different combinations of polymer support material, drug and adhesive.

5. The device of claim 2, wherein the 3-dimensional pattern includes:
a first plurality of printed elements including only polymer support material;
a second plurality of printed elements including only drug; and
a third plurality of printed elements including only adhesive.

6. The device of claim 5, wherein the first plurality of printed elements, the second plurality of printed elements and the third plurality of printed elements have different physical arrangements.

7. The device of claim 2, wherein the 3-dimensional pattern includes first and second pluralities of printed elements, and the printed elements in the first plurality of printed elements have a different physical arrangement than printed elements in the second plurality of printed elements.

8. The device or claim 7, wherein the printed elements in the first and second pluralities of printed elements have different combinations of polymer support material, drug and/or adhesive.

9. The device of claim 2, wherein the 3-dimensional pattern is formed by a plurality of printed elements that have a same physical arrangement.

10. The device or claim 9, wherein each of the plurality of printed elements include a different combination of polymer support material, drug and/or adhesive.

11. The device of claim 2, wherein the 3-dimensional pattern includes first and second pluralities of printed elements, and the printed elements in the first plurality of printed elements are printed in a different layout than printed elements in the second plurality of printed elements.

12. The device of claim 11, wherein the printed elements in the first and second pluralities of printed elements have different combinations of polymer support material, drug and/or adhesive and have different physical arrangements.

13. The device of claim 1, wherein the discrete areas of drug are separated from each other, at least in part, by gaps or voids of the polymer support material and/or adhesive.

14. The device of claim 1, wherein the pattern includes discontinuous elements that each include polymer support material, drug and adhesive.

15. The device of claim 1, wherein the device is arranged to provide minimal, if any, structural support to a body site to which the adhesive adheres the 3-dimensional pattern.

16. The device of claim 1, wherein the polymer support material is formed in a first layer, the drug is formed in a second layer on the first layer, and the polymer support material is formed in a third layer on the second layer, and
wherein the pattern includes voids that separate the discrete areas of drug.

17. The device of claim 16, wherein the adhesive is formed in a fourth layer on the third layer.

18. The device of claim 17, further comprising a backing material formed as a sheet, wherein the first layer is formed on the backing material.

19. The device of claim 1, further comprising:
a solvent mixed with the polymer support material, drug and/or adhesive.

20. The device of claim 1, wherein the 3-dimensional pattern includes a plurality of printed elements that have different combinations of polymer support material, drug, adhesive and/or a solvent.

21. The device of claim 1, further comprising:
a backing material that carries the pattern of polymer support material, drug and adhesive and is arranged to support the polymer support material, drug and adhesive when the device is deployed at a body site.

22. The device of claim 21, wherein the backing material is arranged to degrade faster than the polymer support material and/or adhesive.

23. The device of claim 21, wherein the backing material is arranged to provide structural support to the body site and/or to provide a barrier layer function.

24. The device of claim 1, wherein the polymer support material is degradable, and the drug is associated with the polymer support material such that portions of the drug are released as the polymer support material degrades.

25. A method for providing a delivery device, comprising:
providing a 3-dimensional pattern of polymer support material, drug and adhesive in an arrangement such that the drug is in association with the polymer support material and portions of the drug are released after deployment of the device in a body, and the adhesive is arranged to adhere the pattern to body tissue, wherein the drug is arranged in discrete areas that are separated from each other.

26. The method of claim 25, wherein the step of providing the 3-dimensional pattern comprises:
printing a plurality of printed elements each comprising polymer support material, drug and/or adhesive, the plurality of printed elements forming the 3-dimensional pattern.

27. The method of claim 26, wherein the step of printing a plurality of printed elements comprises:
printing a plurality of printed elements each comprising polymer support material, drug and adhesive, the plurality of printed elements forming the 3-dimensional pattern.

28. The method of claim 26, wherein the step of printing a plurality of printed elements comprises:
printing a plurality of printed elements having a first combination of polymer support material, drug and/or adhesive; and
printing a plurality of printed elements having a second combination of polymer support material, drug and/or adhesive that is different from the first combination.

29. The method of claim 26, wherein the step of printing a plurality of printed elements comprises:
printing a first plurality of printed elements including only polymer support material;
printing a second plurality of printed elements including only drug; and
printing a third plurality of printed elements including only adhesive.

30. The method of claim 29, wherein the first plurality of printed elements, the second plurality of printed elements and the third plurality of printed elements have different physical arrangements with respect to each other.

31. The method of claim 26, wherein at least two printed elements in the 3-dimensional pattern have a different physical arrangement.

32. The method or claim 31, wherein each of the two printed elements includes a different combination of polymer support material, drug and/or adhesive.

33. The method of claim 26, wherein the 3-dimensional pattern is formed by a plurality of printed elements that have a same physical arrangement.

34. The method or claim 33, wherein each of the plurality of printed elements include a different combination of polymer support material, drug and/or adhesive.

35. The method of claim 26, wherein the step of printing a plurality of printed elements comprises:
printing first and second pluralities of printed elements such that the printed elements in the first plurality of printed elements are printed in a different layout than printed elements in the second plurality of printed elements, wherein the printed elements in the first and second pluralities of printed elements have different combinations of polymer support material, drug and/or adhesive and have different physical arrangements.

36. The method of claim 25, wherein the step of providing the 3-dimensional pattern comprises:
printing the adhesive in a desired arrangement of printed elements on a tissue surface of a body; and
providing polymer support material in a desired arrangement of printed elements in association with the adhesive so that the adhesive adheres the polymer support material to the tissue surface.

37. The method of claim 36, further comprising:
providing the drug in a desired arrangement of printed elements in association with the polymer support material while the polymer support material is adhered to the tissue surface.

38. The method of claim 36, wherein the drug is mixed and printed with the polymer support material onto the adhesive printed elements.

39. The method of claim 37, further comprising:
printing the polymer support material and drug in a desired arrangement of printed elements onto a backing material; and
applying the polymer support material and drug with the backing material to the adhesive on the tissue surface.

40. The method of claim 25, wherein the 3-dimensional pattern includes a plurality of voids that separate the discrete areas of drug.

41. The method of claim 25, wherein the 3-dimensional pattern includes a plurality of discontinuous elements of polymer support material and drug.

42. The method of claim 25, further comprising:
mixing a solvent with the polymer support material, drug and/or adhesive to form a mixture, and
forming at least a portion of the 3-dimensional pattern by printing the mixture in a desired arrangement of printed elements.

43. The method of claim 25, wherein the step of providing the 3-dimensional pattern comprises:
forming the pattern on a backing material arranged to support a tissue surface to which the 3-dimensional pattern is adhered.

44. The method of claim 25, wherein the polymer support material is degradable after implant in a body, and the drug is released as the polymer support material degrades.
